(19) United States
(12) Patent Application Publication
(10) Pub. No.: US 2022/0323662 A1
(43) Pub. Date: Oct. 13, 2022

(54) VASCULAR ACCESS DEVICE WITH ARTERIOVENOUS FISTULA SUPPORT

(71) Applicant: VOYAGER BIOMEDICAL, INC., Houston, TX (US)

(72) Inventors: Alan GLOWCZWSKI, College Station, TX (US); Kimberly Marie NEELY, Houston, TX (US)

(21) Appl. No.: 17/766,054

(22) PCT Filed: Oct. 3, 2020
(86) PCT No.: PCT/US2020/054169

§ 371 Date: Apr. 1, 2022

Related U.S. Application Data
(60) Provisional application No. 62/910,984, filed on Oct. 4, 2019.

(51) Int. Cl. A61M 1/36; A61M 1/14

(52) U.S. Cl. CPC A61M 1/3655; A61M 1/14

(57) ABSTRACT

A vascular access device with arteriovenous fistula support includes a top portion and a bottom portion. The top portion has a partial artery channel, a partial vessel channel, and a partial arteriovenous joint between the partial artery channel and the partial vessel channel, where the partial vessel channel has a vascular access aperture for exposing the vessel. The bottom portion has a corresponding partial artery channel, partial vessel channel, and partial arteriovenous joint between the partial artery channel and the partial vessel channel. When the top portion is coupled to the bottom portion, the two partial artery channels couple together to form an artery channel, the two partial vessel channels couple together to form a vessel channel, and the two partial arteriovenous joints couple together to form an arteriovenous joint for an arteriovenous fistula.

### BACKGROUND

Often, when a person loses most of their kidney function, dialysis is required to perform some of the functions of the kidneys. Namely, dialysis removes waste, salt, and excess water from the blood to prevent a toxic build-up in the body. Dialysis also helps to maintain a safe level of chemicals in the blood (e.g., potassium, sodium, and biocarbonate) as well as help control the person's blood pressure. Approximately half a million Americans are on dialysis alone.

The most popular form of dialysis is hemodialysis. In hemodialysis, blood is removed from a patient's blood vessel, ran through a dialysis machine which functions as an artificial kidney, and returned back to the patient's blood vessel. Often, in hemodialysis, an arteriovenous (AV) fistula is created to provide suitable blood pressure and flow in the patient's blood vessel. The AV fistula is a connection between an artery and a vessel, usually created in the arm with a surgical procedure. Surgically created AV fistulas are a preferred mode of vascular access for long-term vascular dialysis treatment and is favored by Medicare over other options. The AV fistula results in increased flow and pressure within a vessel which becomes the access point for dialysis needles. Indeed, the closer the access point in the vessel is to the AV fistula itself, the better the blood flow. After surgery, there is an average 60 day waiting period for the fistula to mature before it can be used.

In approximately 40% of cases, the AV fistula fails to become useable and subsequent surgeries are required. Approximately 33% of cases require transposition surgery, which is needed when the vessel is too deep within the arm to access. After a useable AV fistula is created, the patient and caregivers still endure the challenges of getting a “good stick” each time there is a dialysis treatment, performed at least three times per week. The access vessel must be accurately targeted, sometimes requiring sticks with two 15-gauge needles. Bad punctures are common and, in the most severe cases, may result in significant blood loss and collapse of the fistula (approximately 5.2% patients have significant infiltrations which are a result of a bad puncture and frequently lead to loss of the AV fistula). Furthermore, most AV fistulas only last about three years, largely failing because of excessive and bad punctures. The failed AV fistula site requires additional surgical or radiological intervention to be recovered. If recovery is unsuccessful, a new AV fistula needs to be created in a different location.

In some situations, the increased blood flow through the vessel due to the AV fistula can enlarge the vessel over time, further increasing blood flow through the vessel. Too much blood flow through the AV fistula and into the vessel can leave the outer extremities, which are served by the artery, without enough blood, causing pain and discomfort to the patient. Too much blood flow through the AV fistula and into the vessel can also send too much blood directly to the heart, causing heart issues up to and including heart failure.

### BRIEF SUMMARY

Vascular access devices with arteriovenous fistula support are provided. Arteriovenous fistulas are often needed to supply enough blood flow and pressure to support hemodialysis of a patient's blood. The vascular access device provides a place to draw blood from a vessel that is close to the artery (and therefore has adequate blood flow and pressure to support hemodialysis of a patient's blood) and provides structural support to the vessel, artery, and arteriovenous fistula to prevent damage. Advantageously, the described vascular access device can protect an AV fistula and therefore prevent or at least minimize likelihood of invasive and redundant surgeries, as well as prevent too much blood flow through the AV fistula that causes heart issues and/or not enough blood to get to a patient's outer extremities.

A vascular access device with arteriovenous fistula support can include a top portion and a bottom portion that couple together. The top portion has a partial artery channel for receiving a top half of an artery, a partial vessel channel for receiving a top half of a vessel, and a partial arteriovenous joint between the partial artery channel and the partial vessel channel for receiving a top half of an arteriovenous fistula. The partial vessel channel of the top portion includes a vascular access aperture for exposing the vessel so that blood may be drawn from the vessel. The bottom portion has a partial artery channel for receiving a bottom half of the artery, a partial vessel channel for receiving a bottom half of the vessel, and a partial arteriovenous joint between the partial artery channel and the partial vessel channel for receiving a bottom half of the arteriovenous fistula. When the top portion is coupled to the bottom portion, the partial artery channel of the top portion and the partial artery channel of the bottom portion couple to one another to form an artery channel, the partial vessel channel of the top portion and the partial vessel channel of the bottom portion couple to one another to form a vessel channel, and the partial arteriovenous joint of the top portion and the partial arteriovenous joint of the bottom portion couple to form an arteriovenous joint for an arteriovenous fistula.

In some cases, the partial artery channel of the top portion and the partial artery channel of the bottom portion are semi-cylindrical, and when the top portion is coupled to the bottom portion, the semi-cylindrical artery channel of the top portion and the semi-cylindrical artery channel of the bottom portion couple to one another to form a cylindrical artery channel. In some cases, the partial vessel channel of the top portion and the partial vessel channel of the bottom portion are semi-cylindrical, and when the top portion is coupled to the bottom portion, the semi-cylindrical vessel channel of the top portion and the semi-cylindrical vessel channel of the bottom portion couple to one another to form a cylindrical vessel channel. In some cases, all of the partial artery channels and the partial vessel channels are semi-cylindrical such that both the artery channel and the vessel channel are cylindrical.

In some cases, the partial vessel channel of the bottom portion is elongated relative to the partial vessel channel of the top portion so that as a dialysis needle is inserted into the vessel at an angle relative to a surface of the skin of a patient, the needle does not rupture or damage the back wall of the vessel (i.e., does not go completely through the vessel and out the other side). In some cases, the vascular access aperture is elongated parallel to the vessel of the patient so that as a dialysis needle is inserted into the vessel at an angle relative to a surface of the skin of a patient, the needle does not rupture or damage the back wall of the vessel (i.e., does not go completely through the vessel and out the other side).

In some cases, the surfaces of the top portion and the bottom portion are porous to allow for collagen to form around the top portion and the bottom portion after implantation into a patient. In some cases, a guide lip surrounding the vascular access aperture of the partial vessel channel of the top portion is included to help guide the dialysis needle into the correct position needed for dialysis treatment.

In some implementations, the vessel channel of the vascular access device curves in to the arteriovenous joint while the artery channel of the vascular access device is straight. In some implementations, the artery channel of the vascular access device curves in to the arteriovenous joint while the vessel channel of the vascular access device is straight. In some implementations, both the artery channel and the vessel channel curve in to the arteriovenous joint. In some implementations, both the artery channel and the vessel channel are straight.

In some implementations, an angle between a vertical axis perpendicular to the vascular access aperture of the device and an axis running through center points of the vessel channel and the artery channel is greater than 45 degrees. In some implementations, an angle between a vertical axis perpendicular to the vascular access aperture of the device and an axis running through center points of the vessel channel and the artery channel is between 30 degrees and 45 degrees. In some implementations, an angle between a vertical axis perpendicular to the vascular access aperture of the device and an axis running through center points of the vessel channel and the artery channel is less than 30 degrees.

In some implementations in which an end-to-side AV fistula is created, the vessel channel is perpendicular to the artery channel and the arteriovenous joint is disposed in a position where the artery channel abuts the vessel channel.

A method of using the vascular access device can include creating an incision to insert the vascular access device, positioning the partial artery channel of the bottom portion of the vascular access device around the bottom half of an artery and the partial vessel channel of the bottom portion of the vascular access device around the bottom half of a vessel, positioning the partial artery channel of the top portion of the vascular access device around the top half of the artery and the partial vessel channel of the top portion of the vascular access device around the top half of a vessel to couple the top portion to the bottom portion, closing the incision used to insert the vascular access device and allowing the tissue surrounding the vascular access device to heal, and after the incision is healed, creating an AV fistula between the artery and the vessel without uncoupling the top portion of the vascular access device from the bottom portion of the vascular access device.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A illustrates an angled view of an example vascular access device with side-to-side arteriovenous fistula support.

FIG. 1B illustrates a cross-sectional view of a vascular access device housing a portion of a patient's artery, vessel, and AV fistula.

FIGS. 2A-2D illustrate various embodiments of example vascular access devices with side-to-side arteriovenous fistula support.

FIG. 3A illustrates an exploded view of an example vascular access device with side-to-side arteriovenous fistula support.

FIG. 3B illustrates a cutaway front view of an example vascular access device with side-to-side arteriovenous fistula support.

FIG. 3C illustrates a side view of a top portion of an example vascular access device with side-to-side arteriovenous fistula support.

FIG. 3D illustrates a top view of a bottom portion of an example vascular access device with side-to-side arteriovenous fistula support.

FIG. 3E illustrates a side view of the bottom portion of an example vascular access device with side-to-side arteriovenous fistula support.

FIG. 4 is an image of a vascular access device according to FIGS. 3A-3E that is implanted in a goat.

FIG. 5 is a view of the vessel and the artery after formation of a side-to-side arteriovenous fistula.

FIG. 6A illustrates an angled view of a vascular access device with side-to-side arteriovenous fistula support in which the vessel and artery are not parallel to each other with respect to the surface of the patient's skin.

FIG. 6B illustrates a side view of a vascular access device with side-to-side arteriovenous fistula support in which the vessel and artery are not parallel to each other with respect to the surface of the patient's skin.

FIGS. 6C and 6D illustrate cross-sectional views of vascular access devices with side-to-side arteriovenous fistula support in which the vessel and artery are not parallel to each other with respect to the surface of the patient's skin.

FIG. 6E illustrates a bad puncture in a vascular access device with side-to-side arteriovenous fistula support in which the vessel and artery are not parallel to each other with respect to the surface of the patient's skin.

FIG. 7 illustrates two needles in the vessel, one upstream, one downstream, as they would be during dialysis.

FIG. 8 illustrates an angled view of a vascular access device with two vascular access apertures.

FIGS. 9A and 9B illustrate example vascular access devices with vascular access apertures offset from the AV fistula.

FIG. 10A is a view of the vessel and the artery after formation of an end-to-side arteriovenous fistula.

FIG. 10B illustrates a top view of a vascular access device with end-to-side arteriovenous fistula support.

FIG. 10C illustrates a bottom view of a top portion of a vascular access device with end-to-side arteriovenous fistula support.

FIG. 11 illustrates a top view of a vascular access device with side-to-side arteriovenous fistula support around an artery graft and a vessel.

FIG. 12 illustrates a method of implanting the vascular access device with subsequent creation of an AV fistula.

### DETAILED DESCRIPTION

Vascular access devices with arteriovenous fistula support are provided. Arteriovenous fistulas are often needed to supply enough blood flow and pressure to support hemodialysis of a patient's blood. The vascular access device provides a place to draw blood from the vessel that is close to the artery (and therefore has adequate blood flow and pressure to support hemodialysis of a patient's blood) and provides structural support to the vessel, artery, and arteriovenous fistula to prevent damage. Advantageously, the described vascular access device can protect an AV fistula and therefore prevent or at least minimize likelihood of invasive and redundant surgeries, as well as prevent too much blood flow through the AV fistula that causes heart issues and/or not enough blood to get to a patient's outer extremities.

As used herein, “successful cannulation” or a “good puncture” refers to when a needle/cannula is placed in a vessel to provide vascular access and does not damage any tissue any more than necessary to be placed in the vessel.

As used herein, “unsuccessful cannulation” or a “bad puncture” refers to when a needle/cannula damages more tissue than necessary to be placed in the vessel, whether the needle/cannula is actually placed in the vessel (or not) and/or enters the AV fistula and/or artery. Examples of a bad puncture include when a needle/cannula goes into the vessel and out the back wall of the vessel, or when the vessel is missed altogether, both of which can damage the vessel and/or the tissue surrounding the vessel and contribute to the collapse of the vessel and/or AV fistula, as well as cause blood loss into the surrounding tissue and formation of a hematoma. Another example of a bad puncture includes when a needle/cannula enters the artery, which can be dangerous if any air (e.g., an air bubble) from the needle/cannula enters the artery, which can lead to an air embolism.

As used herein, referring to a portion of the device as “curved” or having “curvature” means that portion of the device has a substantially uniform deviation from straight with respect to a plane perpendicular to the bottom surface of the device.

As used herein, “semi-cylindrical” refers to an object that has a shape of a cylindric section (of a cylindrical surface) where the intersecting plane of the cylinder forms a general shape of a horizontal cylindrical segment. However, the semi-cylindrical shape does not require the cylindric section of the cylindrical surface to appear as being formed from a single plane. For example, the perpendicular-to-the-axis cross-section of the semi-cylindrical shape can appear as a pie shape/sector.

As used herein, “joint” refers to a connection and/or absence of material between the vessel channel and the artery channel that may or may not include structure around the connection and/or absence of material.

As used herein, “side-to-side” refers to a side of a vessel and a side of an artery (or another vessel) that abut and/or come close to one another. As used herein, “end-to-side” refers to an end of a vessel abutting a side of an artery (or another vessel).

As used herein, “artery” may refer to an actual artery or an artery graft.

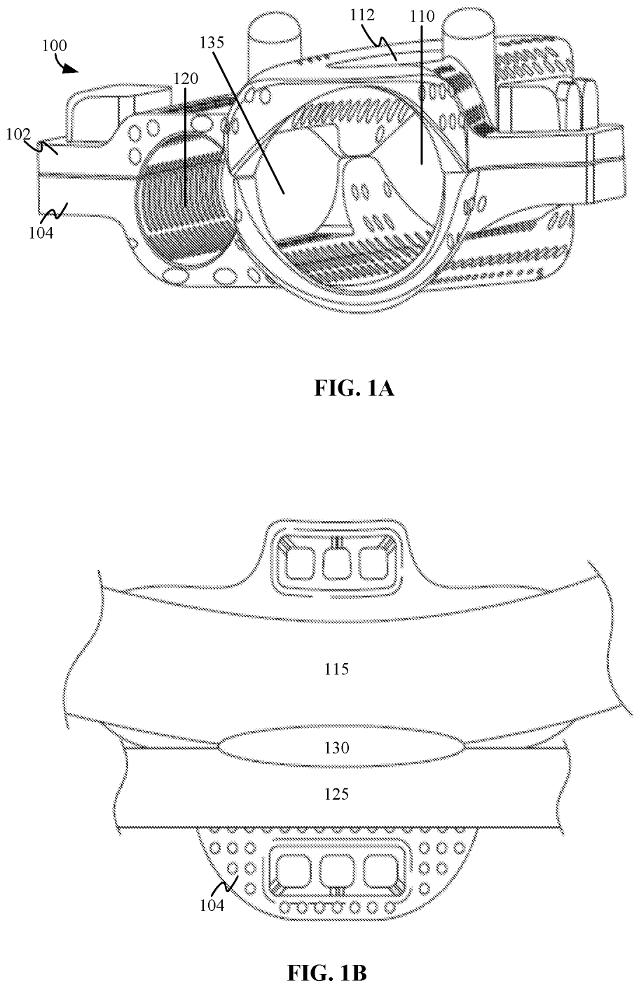

FIG. 1A illustrates an angled view of an example vascular access device with side-to-side arteriovenous fistula support; and FIG. 1B illustrates a cross-sectional view of a vascular access device housing a portion of a patient's artery, vessel, and AV fistula. Referring to FIGS. 1A and 1B, the vascular access device 100 includes a top portion 102 and a bottom portion 104 that are coupled together to form a vessel channel 110 that can encase a vessel 115 and an artery channel 120 that can encase an artery 125. Of course, two vessels may be enclosed in the channels 110, 120 instead of a vessel and an artery should a medical practitioner choose to do so.

The vessel channel 110 provides structural support and external protection for a vessel 115 of a patient. By providing structural support and external protection for the vessel 115 of a patient, the vessel channel 110 prevents a bad puncture (e.g., through a back wall of the vessel 115), which also decreases the amount of trauma incurred to the vessel 115 of the patient and diminishes subsequent scarring and stenosis of the vessel 115. The vessel channel 110 also includes a vascular access aperture 112 that is elongated parallel to the vessel of the patient. The vascular access aperture 112 provides easy access for a dialysis needle to deliver a good puncture to the vessel of the patient and limits the risk of infection by utilizing the skin as a natural barrier to pathogens. Indeed, the vascular access aperture 112 provides a greater surface area for a good puncture than other devices or no devices at all. This enables good punctures in different locations along the surface of the patient's skin, which helps prevent skin breakdown and gives the patient's skin a chance to heal.

The artery channel 120 provides structural support and external protection for an artery 125 of a patient. By providing structural support and external protection for the artery 125 of the patient, the artery channel 120 prevents the artery from being punctured or damaged (e.g., through a bad puncture of the vessel).

An AV fistula 130 can be formed between the vessel 115 and artery 125 and encased in an AV joint 135. The vessel channel 110 and the artery channel 120 can be any shape suitable to house a vessel and artery and therefore, although the channels 110, 120 are illustrated as cylindrical, the cross-section perpendicular to the axis of the cylinder is not required to be a perfect circle or ellipse. Indeed, in other cases, the cross-sectional shape of the channels 110, 120 may be any shape and/or size to fully enclose portions of the vessel 115 and the artery 125 near the AV fistula 130.

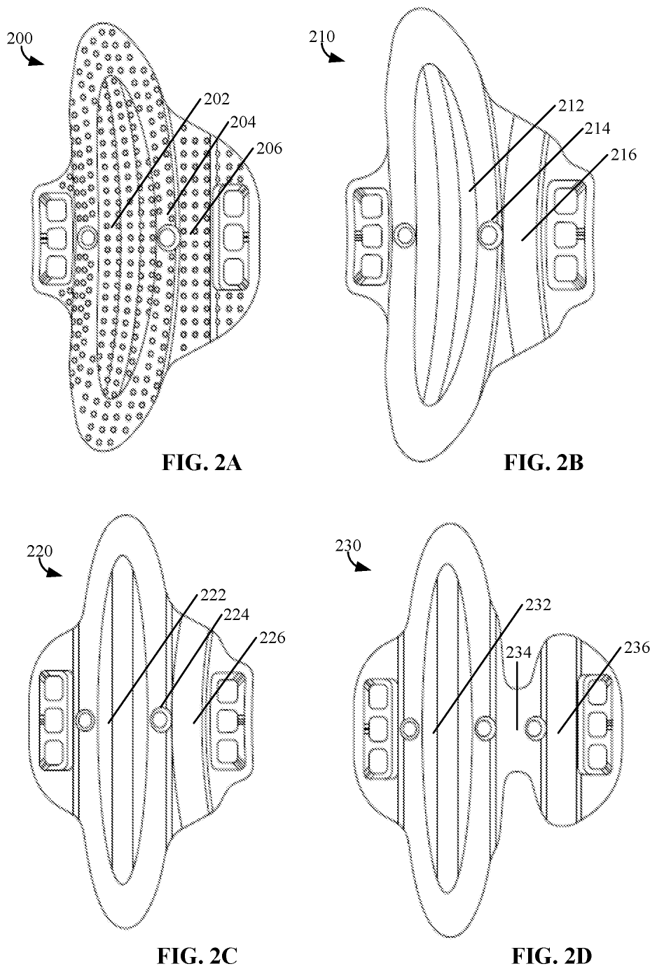

In the illustrated example, the vessel channel 110 curves towards the artery channel 120. However, as illustrated in FIGS. 2A-2D, there are various configurations of straight and curved channels that may be implemented.

In some cases, the surfaces of the device 100 are porous. The pores in the device 100 allow for collagen (i.e., scar tissue) to form around the top portion 102 and the bottom portion after implantation of the device 100 into a patient. The porosity of the device 100 allows ingrowth of fibrovascular tissue, which adheres to the device 100. The fibrovascular tissue allows the body to mount an immune response to any bacteria which are instilled during needle cannulation as well as prevents implantation of bacteria on the surface of the device 100. The porosity of the channels 110, 120 allow collagen to integrate into the vessel and arterial walls, supporting them biologically as well as mechanically. The collagen that forms around the vessel and into the porous channels 110, 120 acts as a scaffold that helps keep the vessel and artery open. The collagen also aids in prevention of collapse of the vessel from repeated sticks and weakening of the vessel wall as well as create a biological seal across the vascular access aperture 112. The porosity of the device 100 may include spaces/holes that can be one nanometer in size up to almost 1 millimeter in size. In some implementations, the pores can be a range of sizes or a specific size, the range or specific size being 100, 150, 200, 250, 300, 350, 400, 450, 500, 550, 600, 650, 700, 750, and/or 800 microns in size. The porosity of the surfaces of the device 100 provides radial support for the vessel of the patient and helps to hold it open during the needle's entry into the vessel. It should be noted that the pores are small enough to prevent the passage of the needle through the surface of the device 100. The surface of the device 100 (having the pores) may be formed by sintering metallic beads (e.g., titanium beads) or powders onto the surface, machining, sandblasting, laser etching, injection molding, and/or 3D printing. It should also be noted that, in some cases, all of the surfaces of the device 100 that are exposed to human tissue are porous.

In some cases, some or all of the device 100 is made of biocompatible plastic, metal, or ceramic, such as titanium, Poly Ether Ether Ketone (PEEK), aluminum oxide, stainless steel, polyvinylchloride and the like. In some cases, some or all of the device 100 is made of radiolucent and/or radiopaque materials. This can help a surgeon if any subsequent procedures are needed (e.g., creation of the AV fistula subsequent to the implantation of the device 100). For example, radiolucent and/or radiopaque markers may be fixed to the device 100 so that surgeons and/or interventionalists who are implanting/revising/accessing the device 100 can see the device under various imaging modalities. The radiolucent/radiopaque markers can also be utilized to identify appropriate sites for withdrawal and/or injection of fluid.

FIGS. 2A-2D illustrate various embodiments of example vascular access devices with side-to-side arteriovenous fistula support. FIG. 2A illustrates a vascular access device 200 where the vessel channel 202 of the vascular access device 200 curves in to the arteriovenous joint 204 while the artery channel 206 of the vascular access device 200 is straight. FIG. 2B illustrates a vascular access device 210 where both the vessel channel 212 and the artery channel 216 curve in to the arteriovenous joint 214. FIG. 2C illustrates a vascular access device 220 the artery channel 226 of the vascular access device 220 curves in to the arteriovenous joint 224 while the vessel channel 222 of the vascular access device 220 is straight. FIG. 2D illustrates a vascular access device 230 where both the vessel channel 232 and the artery channel 236 are straight with the arteriovenous joint 234 supported therebetween. This embodiment may be advantageous in a situation in which the artery and the vessel are already close enough to one another to create the AV fistula without moving either the vessel or the artery (e.g., 5 to 10 millimeters).

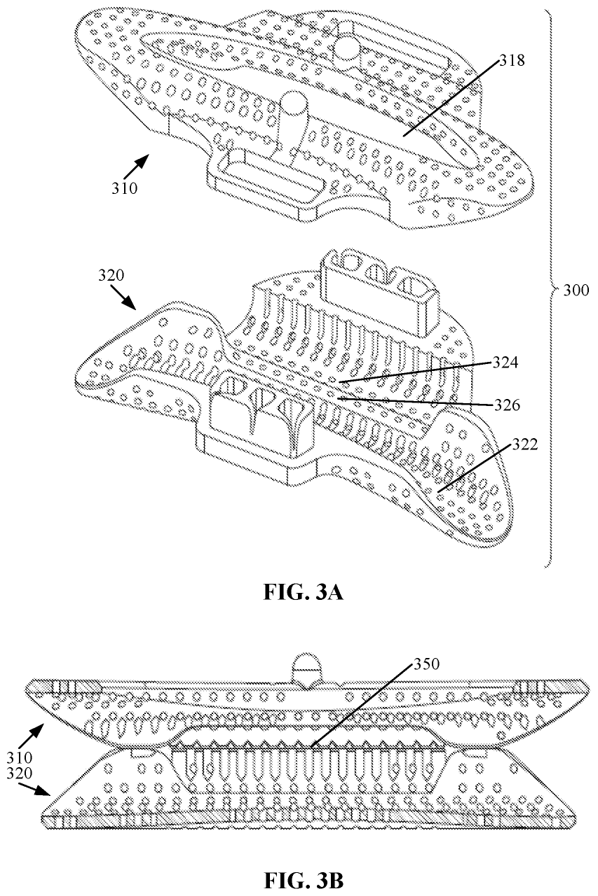

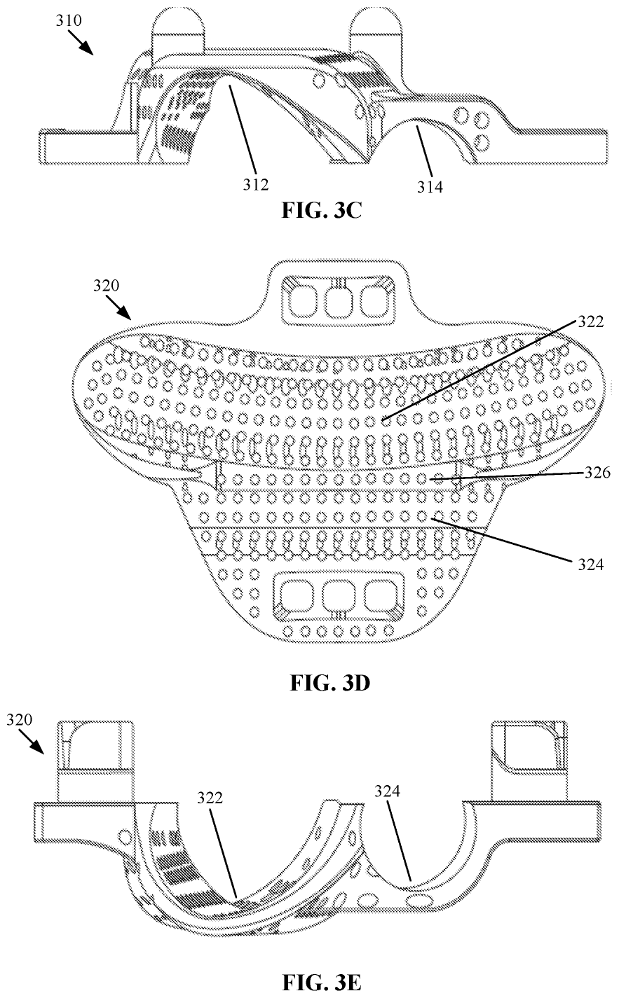

FIG. 3A illustrates an exploded view of an example vascular access device with side-to-side arteriovenous fistula support; FIG. 3B illustrates a cutaway front view of an example vascular access device with side-to-side arteriovenous fistula support; FIG. 3C illustrates a side view of a top portion of an example vascular access device with side-to-side arteriovenous fistula support; FIG. 3D illustrates a top view of a bottom portion of an example vascular access device with side-to-side arteriovenous fistula support; FIG. 3E illustrates a side view of the bottom portion of an example vascular access device with side-to-side arteriovenous fistula support; and FIG. 4 is an image of a vascular access device according to FIGS. 3A-3E that is implanted in a goat. Referring to FIG. 3A, a vascular access device 300 includes a top portion 310 and a bottom portion 320. As can be seen in FIGS. 3A and 3C, the top portion 310 includes a partial vessel channel 312 for receiving a top half of a vessel, a partial artery channel 314 for receiving a top half of an artery, a partial arteriovenous joint (not shown in the view), and a vascular access aperture 318. As can be seen in FIGS. 3A, 3D and 3E, the bottom portion 320 includes a partial vessel channel 322 for receiving a bottom half of a vessel, a partial artery channel 324 for receiving a bottom half of an artery, and a partial arteriovenous joint 326. The partial vessel channel 312, partial artery channel 314, and a partial arteriovenous joint (not shown) of the top portion 310 correspond to the partial vessel channel 322, partial artery channel 324, and partial arteriovenous joint 326 of the bottom portion 320. These partial channels form a vessel channel (e.g., a vessel channel 330 of FIG. 4) and an artery channel (e.g., artery channel 340 of FIG. 4) when the top portion 310 is coupled to the bottom portion 320. In addition, the partial arteriovenous joints form an arteriovenous joint (see arteriovenous joint 350 of FIG. 3B) when the top portion 310 is coupled to the bottom portion 320.

In some cases, the partial vessel channel 312 of the top portion 310 and the partial vessel channel 322 of the bottom portion 320 are semi-cylindrical. Therefore, when the top portion 310 is coupled to the bottom portion 320, the partial vessel channel 312 of the top portion 310 and the partial vessel channel 322 of the bottom portion 320 couple to one another to form a cylindrical vessel channel. In some cases, the partial artery channel 314 of the top portion 310 and the partial artery channel 324 of the bottom portion 320 are semi-cylindrical. Therefore, when the top portion 310 is coupled to the bottom portion 320, the partial artery channel 314 of the top portion 310 and partial artery channel 324 of the bottom portion 320 couple to one another to form a cylindrical artery channel. In some cases, all of the partial vessel channels 312, 322 and partial artery channels 314, 324 are semi-cylindrical such that both the artery channel and the vessel channel are cylindrical.

As illustrated in FIG. 3B, the arteriovenous joint 350 is formed when the top portion 310 and the bottom portion 320 couple to one another. When implanted, the arteriovenous joint 350 provides structural support around an arteriovenous fistula. The arteriovenous joint 350 can protect the fistula from being punctured or otherwise damaged from inserting needles. The arteriovenous joint 350 may be any suitable shape, so long as that shape allows for blood to pass from the artery to the vessel through the arteriovenous fistula.

Furthermore, because the punctures (via the vascular access aperture) are so close to the AV fistula, a very high blood flow (e.g., approximately 400 ml/min) and pressure (compared to standard hemodialysis through a vessel relatively far away from an AV fistula) is provided. This allows patients and/or technicians performing the hemodialysis to reduce the capacity needed of a pump or possibly eliminate the pump that is required for normal hemodialysis. That is because the blood is naturally pressurized and pumped by the heart of the patient in an artery more than a vessel that is not connected to an artery via an AV fistula. In other words, the blood flow and pressure of a vessel that is directly connected to an AV fistula and is relatively close to that AV fistula will have greater blood flow and pressure than a vessel without an AV fistula or even a vessel with an AV fistula that is not relatively close to that AV fistula.

With reference to both FIGS. 3A and 4, when implanting the vascular access device 300, the bottom portion 320 is positioned distal to the surgeon implanting the device 300, with the partial vessel channel 322 of the bottom portion 320 being placed around a bottom portion of the vessel 331 of the patient and the partial artery channel 324 being placed around a bottom portion of the artery 341 of the patient. The top portion 310 is positioned proximal to the surgeon implanting the device 300 such that the vascular access aperture 318 is disposed parallel to the surface of the patient's skin (and is accessible). Once implanted, the top portion 310 may be coupled to the bottom portion 320 by any means available and within standards of those required by surgeons.

Advantageously, when the top portion 310 is coupled to the bottom portion 320, the structure of the top portion 310 and the bottom portion 320 around the vessel prevents or at least minimizes the chance of a bad puncture by preventing the needle from going somewhere unintended in the patient. In addition, the porous surface of the device 300 enables the device 300 to integrate into the surrounding tissues which allows the body to mount an immune response to any bacteria which are instilled during needle cannulation. Having the device integrate into the surrounding tissues removes bare surfaces for bacteria to grow on and develop a biofilm.

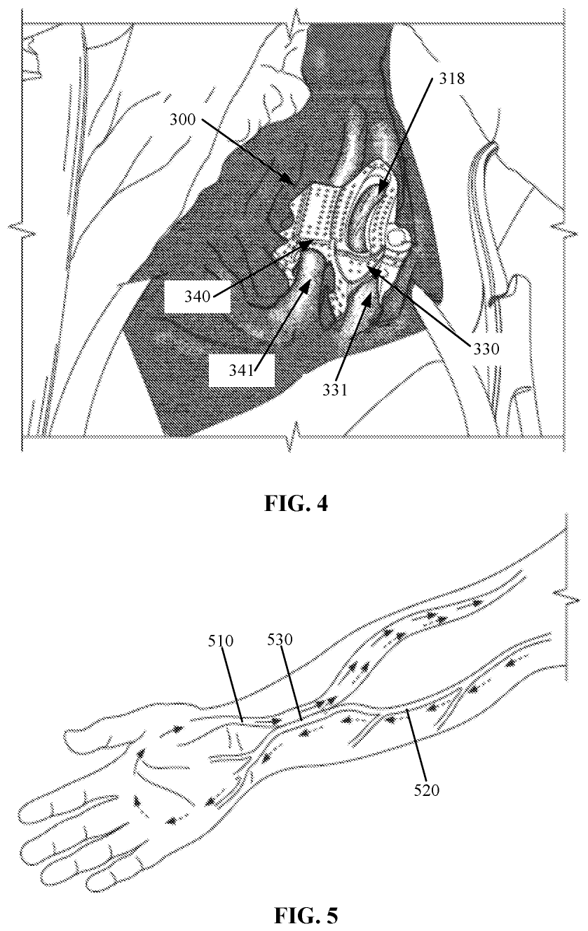

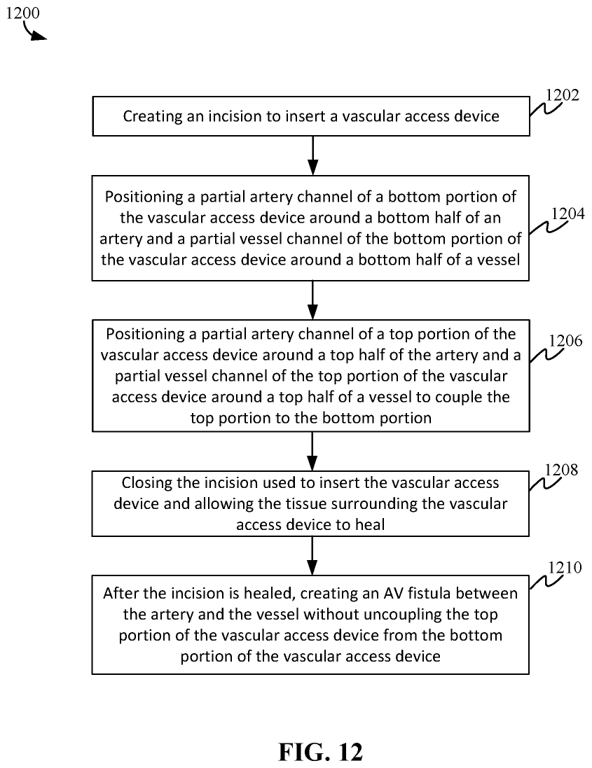

FIG. 5 is a view of the vessel and the artery after formation of a side-to-side arteriovenous fistula. The device (not shown in this figure) surrounds the vessel 510 and the artery 520. During (or before or after) implantation of the device, the AV fistula 530 is created. Indeed, the device may be implanted around an existing AV fistula (e.g., in order to “save” an existing AV fistula). In other cases, the device may also be implanted and the AV fistula created after the device is implanted (e.g., in order to allow the device time to adhere to the surrounding tissue; see corresponding method with respect to FIG. 12). In yet other cases, the device may be implanted when the AV fistula is created (e.g., during the same procedure). In any case, the AV fistula 530 is created and may be completely surrounded by the device so that a dialysis needle (or any other type of needle) cannot penetrate the AV fistula 530. This allows a needle to provide a good puncture to the vessel 510 of the patient (e.g., through the vascular access aperture of the device) without worry that the AV fistula 530 will be damaged. Furthermore, as explained above and below, the device also completely surrounds the artery 520 and all of the vessel 510 except the portion exposed through the vascular access aperture of the device, which is the portion of the vessel 510 that is closest to the surface of the patient's skin.

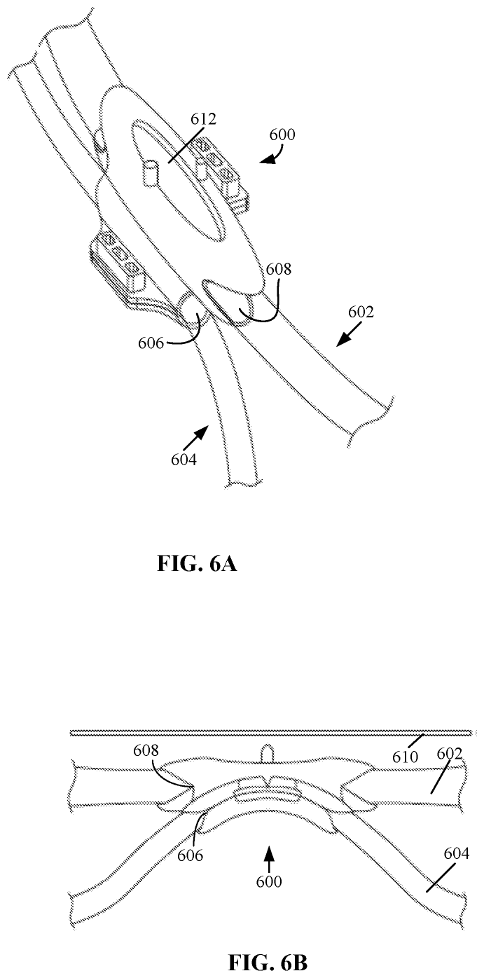

In some patients, depending on the part of the body that the AV fistula 530 is created and the physiology of the individual patient, the vessel 510 may be proximal to the surface of the patient's skin and the artery 520 may be distal from the surface of the patient's skin (i.e., the artery 520 is beneath the vessel 510 in relation to the surface of the patient's skin). Therefore, as explained below with respect to FIGS. 6A-6E, placement of the vascular access device may be disposed such that a vascular access aperture of the top portion is in the most proximal position to the surface of the skin of the patient as allowed for by the patient's physiology. In other words, the position of the vascular access aperture of the vascular access device can be individualized for each patient so that the least invasive method of implanting the device and/or convenient position of the vessel and artery may be utilized to position the device around the vessel and the artery of the patient.

FIG. 6A illustrates an angled view of a vascular access device with side-to-side arteriovenous fistula support in which the vessel and artery are not parallel to each other with respect to the surface of the patient's skin; FIG. 6B illustrates a side view of a vascular access device with side-to-side arteriovenous fistula support in which the vessel and artery are not parallel to each other with respect to the surface of the patient's skin. As can be seen in FIG. 6A, the device 600 encloses a portion of a vessel 602 and a portion of an artery 604 on both sides leading up to an AV fistula. The artery 604 is proximal to the surface of the patient's skin (not shown in this figure) and the vessel 602 is distal to the surface of the patient's skin. Therefore, the device 600 provides an artery channel 606 that is positioned below and to the side of the vessel channel 608.

As can be seen in FIG. 6B, the vessel 602 is positioned relatively close/proximal to the surface of the patient's skin 610 (allowing for a good puncture through the vascular access aperture 612), while the artery 604 is positioned further below/distal to the surface of the patient's skin 610. Indeed, in this example, the artery 604 was transposed (e.g., during implantation of the device 600) closer to the surface of the patient's skin 610, but in a minimal way because the artery channel 606 is below and/or offset from the vessel channel 608. In this way, the surgeon that performed the transposition surgery for the artery 604 did not have to move the artery 604 as far in a vertical direction with respect to the surface of the patient's skin 610 as would have been required had the artery channel 606 and the vessel channel 608 been parallel to each other with respect to the surface of the patient's skin 610. This also provides another advantage to a patient because the surgeon performing the transposition surgery does not have to move as much of the artery 604 in the horizontal direction with respect to the surface of the patient's skin 610 to prevent kinking of the artery 604 as would have been required had the artery channel 606 and the vessel channel 608 been parallel to each other with respect to the surface of the patient's skin 610. It should be understood that, in some situations in which the artery 604 is deeper underneath the patient's skin 610 than the vessel 602, because of the offset between the artery channel 606 and the vessel channel 608 in this embodiment, a transposition surgery may not be required at all.

FIGS. 6C and 6D illustrate cross-sectional views of vascular access devices with side-to-side arteriovenous fistula support in which the vessel and artery are not parallel to each other with respect to the surface of the patient's skin. In FIG. 6C, the angle 620 between a vertical axis 622 perpendicular to the vascular access aperture 624 of the device 626 and an axis 628 running through the center points 630, 632 of the vessel channel 634 and the artery channel 636 is 45 degrees. As can be seen, the angle 620 is sufficiently large to prevent a bad puncture.

In FIG. 6D, the angle 640 between a vertical axis 642 perpendicular to the vascular access aperture 644 of the device 646 and an axis 648 running through the center points 650, 652 of the vessel channel 654 and the artery channel 656 is 30 degrees. As can be seen, the angle 640 is not sufficiently large enough to prevent a bad puncture (e.g., as illustrated below in FIG. 6E). Therefore, a technician or patient utilizing this device may use a needle that cannot go deep enough to reach the AV fistula 658/artery channel 656 (e.g., because of an external device or shortened length of the needle) or take extra care to not insert a needle deep enough to create a bad puncture. In some cases, an external device (not shown) may be used to position the needle for insertion into the vessel channel 654 at an angle and/or length that will not result in a bad puncture.

It should be understood that the angles 620, 640 illustrated in FIGS. 6C and 6D are only examples related to embodiments illustrated in these figures. Furthermore, a range of angles that are not sufficiently large enough to prevent a bad puncture may vary between vascular access devices, as a sufficient angle is also a function of the width of the vascular access aperture. Therefore, in other embodiments, angle 640 of FIG. 6D may be sufficiently large to prevent a bad puncture.

FIG. 6E illustrates a bad puncture in a vascular access device with arteriovenous fistula support in which the vessel and artery are not parallel to each other with respect to the surface of the patient's skin. As can be seen in FIG. 6E, the bad puncture is created when a needle 670 enters the device 672 through the vascular access aperture 674, continues through the vessel channel 676 and the AV fistula 678, and enters the artery channel 680. This is a bad puncture because when the needle 670 enters the artery channel 680, if any air (e.g., an air bubble) from the needle 670 enters the artery 682, an air embolism may be created, which can block the flow of blood further downstream from the AV fistula 678, causing poor circulation to the distal extremity. It should also be understood that in all cases, vascular access devices prevent bad punctures where the needle goes out of an end of the vessel channel.

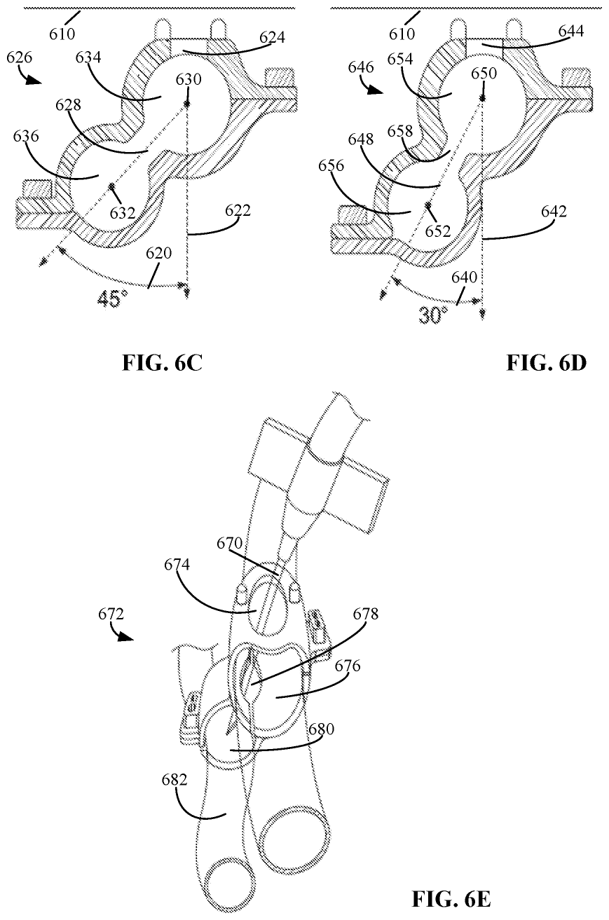

FIG. 7 illustrates two needles in the vessel, one upstream, one downstream, as they would be during dialysis. As can be seen, the upstream needle 702 and the downstream needle 704 are each inserted through the vascular access aperture 706 of the device 700 and into the vessel 708 of the patient. The needles 702, 704 may be taped down to the patient's skin after insertion into the vessel 708 or may be left in the position shown in FIG. 7, depending on the preferences of the patient and/or technician administering the dialysis treatment. Once inserted, the upstream needle 702 “pulls” blood from a high-pressure location in the vessel 708 adjacent to the AV fistula. The downstream needle 704 is inserted into a location in the vessel 708 downstream from the upstream needle 702. It is important to note that the upstream needle 702 withdraws the blood of the patient, which is cleaned in a dialysis machine, and the cleaned blood is returned to the patient's vessel 708 via the downstream needle 704. The return of the cleansed blood downstream from the withdrawal of the (dirty) blood prevents recirculation of the patient's blood. Recirculation of the patient's blood is very inefficient and results in the dialysis taking a lot longer to complete because at least part the already cleaned blood would be re-cleaned in the dialysis machine in an essentially endless loop. Therefore, the dialysis machine would not be cleaning as much of the (dirty) blood as possible because the dialysis machine can only clean so much blood at a time.

Furthermore, because the needles 702, 704 withdraw blood (via the vascular access aperture) so close to the AV fistula, a very high blood flow and pressure (compared to standard hemodialysis through a vessel relatively far away from an AV fistula) is provided. This allows patients and/or technicians performing the dialysis to reduce the capacity needed of a pump or possibly eliminate the pump that is required for normal hemodialysis treatment. That is because the blood is naturally pressurized and pumped by the heart of the patient in an artery 710 more than a normal vessel; the vessel 708 is directly connected to the artery 710 through an AV fistula, thus creating pressure and flow in the vessel 708 that is similar to that of the artery 710.

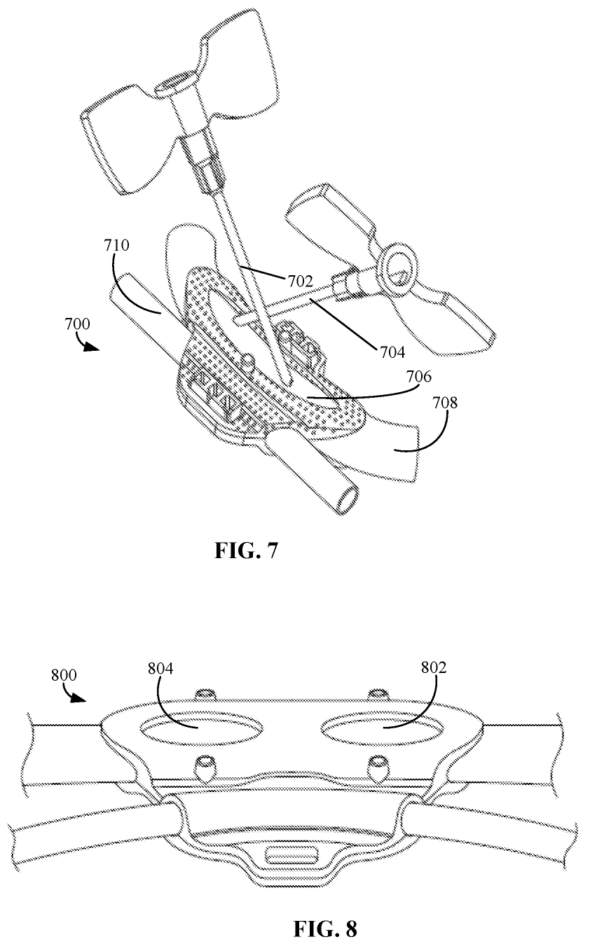

FIG. 8 illustrates an angled view of a vascular access device with two vascular access apertures. As can be seen, the device 800 includes an upstream vascular access aperture 802 and a downstream vascular access aperture 804. This design may be especially useful in cases where an external device and/or marking is used to identify each of the vascular access apertures 802, 804 underneath the skin of the patient. By having separate vascular access apertures 802, 804 for the upstream and downstream needles (e.g., needles 702 and 704 of FIG. 7), a patient and/or technician administering the dialysis treatment may be less inclined to mix the needles up (and therefore preventing recirculation). Another advantage for two vascular access apertures 802, 804 is that patients may have more confidence that the dialysis treatment is being performed correctly, which is often an impediment to a patient administering in-home dialysis treatment themselves.

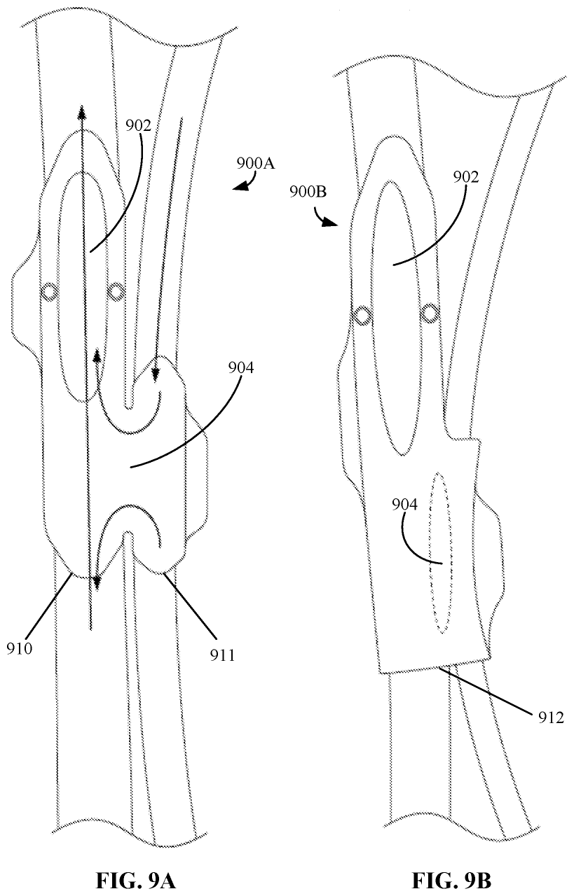

FIGS. 9A and 9B illustrate vascular access devices with vascular access apertures offset from the arteriovenous joints. FIG. 9A shows an arcuate channel opening (910, 911) for each channel; and FIG. 9B shows an alternative opening (912) configuration. As can be seen, each device 900A, 900B includes a vascular access aperture 902 that is downstream and offset from the arteriovenous joint 904. The vascular access aperture 902 and AV joint 904 can be offset such that there is no cross-sectional overlap.

The offset design may be useful in cases where damage to the AV fistula is of great concern to the physician and/or patient. Indeed, because the vascular access aperture 902 is offset from the AV fistula/arteriovenous joint 904, the likelihood of damage to the AV fistula from a bad puncture is further reduced. Furthermore, because of the reduced likelihood of damage to the AV fistula from a bad puncture, patients may have more confidence in administering in-home dialysis treatment themselves. It should also be understood that, except for that which is inconsistent with the apparatus and techniques described with respect to FIGS. 9A and 9B, the same features described with respect to FIGS. 1A through 8 may be present in the devices 900A, 900B illustrated in FIGS. 9A and 9B.

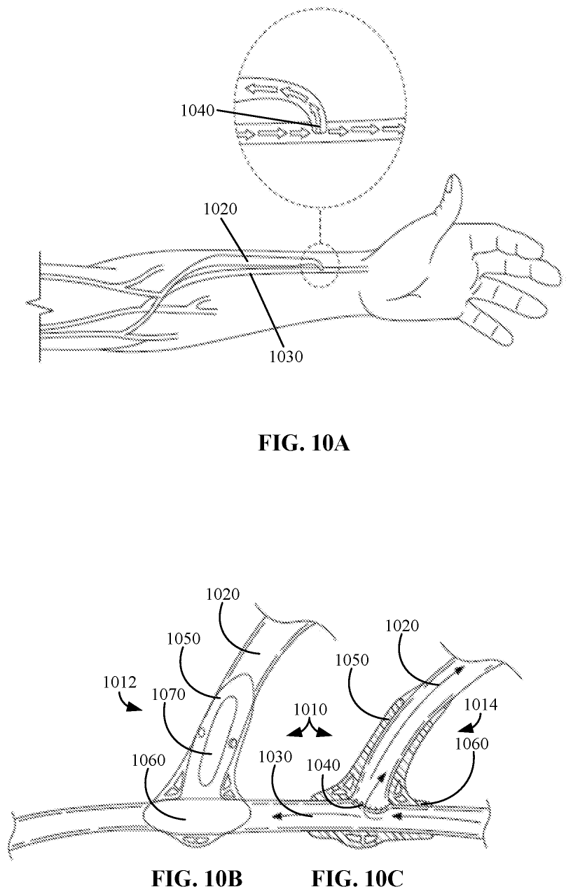

FIG. 10A is a view of the vessel and the artery after formation of an arteriovenous fistula; FIG. 10B illustrates a top view of a vascular access device with end-to-side arteriovenous fistula support; FIG. 10C illustrates a bottom view of a top portion of a vascular access device with end-to-side arteriovenous fistula support. The vascular access device 1010 surrounds the vessel 1020 and the artery 1030. In this embodiment, during (or before or after) implantation of the device 1010, the AV fistula 1040 is created by severing the vessel 1020 and attaching it directly to the artery 1030. As can be seen, the vessel 1020 attaches to the artery 1030 in an approximately perpendicular manner, and therefore the device 1010 is T-shaped (as opposed to H-shaped, X-shaped, or K-shaped as described in the FIGS. 2A-2D). Here, the vessel channel is perpendicular to the artery channel and the arteriovenous joint is disposed in a position where the artery channel abuts the vessel channel.

In any case, the AV fistula 1040 is created and may be completely surrounded by the device 1010 so that a dialysis needle (or any other type of needle) cannot penetrate the AV fistula 1040. This allows a needle to provide a good puncture to the vessel 1020 of the patient through the vascular access aperture 1070 of the device 1010 without worry that the AV fistula 1040 will be damaged.

During implantation of the device 1010, the top portion 1012 and the bottom portion 1014 of the device 1010 are coupled together to form a vessel channel 1050 that can encase the vessel 1020 and an artery channel 1060 that can encase the artery 1030. The vessel channel 1050 provides structural support and external protection for a vessel 1020 of a patient. By providing structural support and external protection for the vessel 1020 of the patient, the vessel channel 1050 prevents a bad puncture, which also decreases the amount of trauma to the vessel 1020 of the patient and diminishes subsequent scarring and stenosis of the vessel 1020. The vessel channel 1050 also includes a vascular access aperture 1070 that is elongated parallel to the vessel 1020 of the patient. The vascular access aperture 1070 provides easy access for a dialysis needle to deliver a good puncture to the vessel 1020 of the patient and limits the risk of infection by utilizing the skin as a natural barrier to pathogens. Indeed, the vascular access aperture 1070 provides a greater surface area for a good puncture than other devices or no devices at all. This enables good punctures in different locations along the surface of the patient's skin, which helps prevent skin breakdown and gives the skin a chance to heal.

The artery channel 1060 provides structural support and external protection for the artery 1030 of a patient. By providing structural support and external protection for the artery 1030 of the patient, the artery channel 1060 prevents the artery 1030 from being punctured or damaged (e.g., through a bad puncture of the vessel 1020).

An AV fistula 1040 can be formed through a connection of the end of the vessel 1020 and a side of the artery 1030. The AV fistula 1040 is fully encased by the device 1010. The vessel channel 1050 and the artery channel 1060 can be any shape suitable to house a vessel 1020 and an artery 1030. Therefore, although the channels 1050, 1060 are illustrated as cylindrical, the cross-section perpendicular to the axis of the cylinder is not required to be a perfect circle or ellipse. Indeed, in other cases, the cross-sectional shape of the channels 1050, 1060 may be any shape and/or size to fully enclose portions of the vessel 1020 and the artery 1030 near the AV fistula 1040.

In some cases, the surfaces of the device 1010 are porous. As explained above with respect to FIGS. 1A and 1B, the pores in the device 1010 allow for collagen (i.e., scar tissue) to form around the top portion 1012 and the bottom portion 1014 after implantation of the device 1010 into a patient. In some cases, all of the surfaces of the device 1010 that are exposed to human tissue are porous.

It should be understood that, except for that which is inconsistent with the apparatus and techniques described with respect to FIGS. 10A-10C, the same features described with respect to FIGS. 1A through 9B may be present in the device illustrated in FIGS. 10B and 10C. It should also be noted that the blood in the artery 1030 flows into the vessel 1020, as illustrated in FIGS. 10A and 10C.

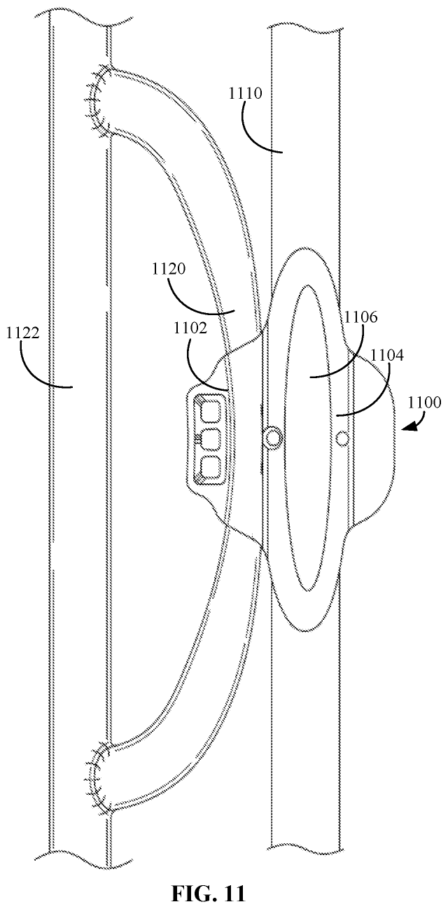

FIG. 11 illustrates a top view of a vascular access device with side-to-side arteriovenous fistula support around an artery graft and a vessel. Similar to vascular access devices described above, the vascular access device 1100 includes an artery channel 1102 and a vessel channel 1104 having a vascular access aperture 1106. Specifically, a top portion and a bottom portion of the vascular access device 1100 are coupled together to form the vessel channel 1104 that encases a vessel 1110 and the artery channel 1102 that encases an artery graft 1120. In this case, the artery graft 1120 is attached at to the artery 1122 at an upstream point and a downstream point so that some of the blood flow from the artery 1122 will flow into the artery graft 1120 at the upstream point and return to the artery 1122 at the downstream point. Because the artery graft 1120 does not have as much blood flow as the artery 1122 would have by itself, the use of the artery graft 1120 can further reduce potential issues that cause heart problems and/or not enough blood to get to a patient's outer extremities while the vascular access device 1100 still provides structural support to the vessel, artery, and arteriovenous fistula to prevent damage. In some cases, the artery graft 1120 is made of polytetrafluoroethylene (PFTE). In some cases, the artery graft 1120 is made of vessels that are surgically removed from other parts of the body. It should be understood that the artery graft 1120 may be made of any material that is safe for use as an artificial vessel.

FIG. 12 illustrates a method of implanting the vascular access device with subsequent creation of an AV fistula. Referring to FIG. 12, a method 1200 includes creating 1202 an incision to insert the vascular access device, positioning 1204 the partial artery channel of the bottom portion of the vascular access device around the bottom half of an artery and the partial vessel channel of the bottom portion of the vascular access device around the bottom half of a vessel, positioning 1206 the partial artery channel of the top portion of the vascular access device around the top half of the artery and the partial vessel channel of the top portion of the vascular access device around the top half of a vessel to couple the top portion to the bottom portion, closing 1208 the incision used to insert the vascular access device and allowing the tissue surrounding the vascular access device to heal, and after the incision is healed, creating 1210 an AV fistula between the artery and the vessel without uncoupling the top portion of the vascular access device from the bottom portion of the vascular access device.

Certain aspects of the invention provide the following non-limiting embodiments:

Example 1. A vascular access device comprising a top portion having a partial artery channel for receiving a top half of an artery, a partial vessel channel for receiving a top half of a vessel, and a partial arteriovenous joint between the partial artery channel and the partial vessel channel, the partial vessel channel having a vascular access aperture for exposing the vessel; and a bottom portion having a partial artery channel for receiving a bottom half of the artery, a partial vessel channel for receiving a bottom half of the vessel, and a partial arteriovenous joint between the partial artery channel and the partial vessel channel; wherein when the top portion is coupled to the bottom portion: the partial artery channel of the top portion and the partial artery channel of the bottom portion couple to one another to form an artery channel; the partial vessel channel of the top portion and the partial vessel channel of the bottom portion couple to one another to form a vessel channel; and the partial arteriovenous joint of the top portion and the partial arteriovenous joint of the bottom portion couple to form an arteriovenous joint for an arteriovenous fistula.

Example 2. The vascular access device of Example 1, wherein the partial artery channel of the top portion and the partial artery channel of the bottom portion are semi-cylindrical; wherein when the top portion is coupled to the bottom portion, the semi-cylindrical artery channel of the top portion and the semi-cylindrical artery channel of the bottom portion couple to one another to form a cylindrical artery channel.

Example 3. The vascular access device of Examples 1 or 2, wherein the partial vessel channel of the top portion and the partial vessel channel of the bottom portion are semi-cylindrical; wherein when the top portion is coupled to the bottom portion, the semi-cylindrical vessel channel of the top portion and the semi-cylindrical vessel channel of the bottom portion couple to one another to form a cylindrical vessel channel.

Example 4. The vascular access device of any of the preceding Examples, wherein the partial vessel channel of the bottom portion is elongated relative to the partial vessel channel of the top portion.

Example 5. The vascular access device of any of the preceding Examples, wherein the vascular access aperture is elongated parallel to a length of the vessel of a patient.

Example 6. The vascular access device of any of the preceding Examples, wherein surfaces of the top portion and the bottom portion are porous to allow for collagen to form around the top portion and the bottom portion after implantation.

Example 7. The vascular access device of any of the preceding Examples, wherein the vessel channel is curved towards the arteriovenous joint.

Example 8. The vascular access device of any of the preceding Examples, wherein the artery channel is curved towards the arteriovenous joint.

Example 9. The vascular access device of any of Examples 1-6, wherein the vessel channel extends perpendicular to the artery channel; and wherein the arteriovenous joint is disposed in a position where the artery channel abuts the vessel channel.

Example 10. The vascular access device of any of the preceding Examples, wherein an angle between a vertical axis perpendicular to the vascular access aperture and an axis running through center points of the vessel channel and the artery channel is greater than 45 degrees.

Example 11. The vascular access device of any of Examples 1-9, wherein an angle between a vertical axis perpendicular to the vascular access aperture and an axis running through center points of the vessel channel and the artery channel is between 30 degrees and 45 degrees.

Example 12. The vascular access device of any of Examples 1-9, wherein an angle between a vertical axis perpendicular to the vascular access aperture and an axis running through center points of the vessel channel and the artery channel is less than 30 degrees.

Example 13. The vascular access device of any of the preceding Examples, wherein the arteriovenous joint is offset from the vascular access aperture.

Example 14. A method of using the vascular access device of any of the preceding Examples includes creating an incision to insert the vascular access device, positioning the partial artery channel of the bottom portion of the vascular access device around the bottom half of the artery and the partial vessel channel of the bottom portion of the vascular access device around the bottom half of the vessel, positioning the partial artery channel of the top portion of the vascular access device around the top half of the artery and the partial vessel channel of the top portion of the vascular access device around the top half of the vessel to couple the top portion to the bottom portion, closing the incision used to insert the vascular access device and allowing the tissue surrounding the vascular access device to heal, and after the incision is healed, creating an AV fistula between the artery and the vessel without uncoupling the top portion of the vascular access device from the bottom portion of the vascular access device.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A vascular access device comprising:
a top portion having a partial artery channel for receiving a top half of an artery, a partial vessel channel for receiving a top half of a vessel, and a partial arteriovenous joint between the partial artery channel and the partial vessel channel, the partial vessel channel having a vascular access aperture for exposing the vessel; and
a bottom portion having a partial artery channel for receiving a bottom half of the artery, a partial vessel channel for receiving a bottom half of the vessel, and a partial arteriovenous joint between the partial artery channel and the partial vessel channel;
wherein when the top portion is coupled to the bottom portion:
the partial artery channel of the top portion and the partial artery channel of the bottom portion couple to one another to form an artery channel;
the partial vessel channel of the top portion and the partial vessel channel of the bottom portion couple to one another to form a vessel channel; and
the partial arteriovenous joint of the top portion and the partial arteriovenous joint of the bottom portion couple to form an arteriovenous joint for an arteriovenous fistula.

2. The vascular access device of claim 1, wherein the partial artery channel of the top portion and the partial artery channel of the bottom portion are each semi-cylindrical; and
wherein when the top portion is coupled to the bottom portion, the semi-cylindrical partial artery channel of the top portion and the semi-cylindrical partial artery channel of the bottom portion couple to one another to form a cylindrical artery channel.

3. The vascular access device of claim 1, wherein the partial vessel channel of the top portion and the partial vessel channel of the bottom portion are each semi-cylindrical; and
wherein when the top portion is coupled to the bottom portion, the semi-cylindrical partial vessel channel of the top portion and the semi-cylindrical partial vessel channel of the bottom portion couple to one another to form a cylindrical vessel channel.

4. The vascular access device of claim 1, wherein the partial artery channel of the top portion, the partial artery channel of the bottom portion, the partial vessel channel of the top portion, and the partial vessel channel of the bottom portion are each semi-cylindrical;
wherein when the top portion is coupled to the bottom portion, the semi-cylindrical partial artery channel of the top portion and the semi-cylindrical partial artery channel of the bottom portion couple to one another to form a cylindrical artery channel, and the semi-cylindrical partial vessel channel of the top portion and the semi-cylindrical partial vessel channel of the bottom portion couple to one another to form a cylindrical vessel channel.

5. The vascular access device of claim 1, wherein the partial vessel channel of the bottom portion is elongated relative to the partial vessel channel of the top portion.

6. The vascular access device of claim 1, wherein the vascular access aperture is elongated parallel to a length of the vessel of a patient.

7. The vascular access device of claim 1, wherein surfaces of the top portion and the bottom portion are porous to allow for collagen to form around the top portion and the bottom portion after implantation.

8. The vascular access device of claim 1, wherein the vessel channel is curved towards the arteriovenous joint.

9. The vascular access device of claim 1, wherein the artery channel is curved towards the arteriovenous joint.

10. The vascular access device of claim 1, wherein both the vessel channel and the artery channel are curved towards the arteriovenous joint.

11. The vascular access device of claim 1, wherein the vessel channel extends perpendicular to the artery channel; and
wherein the arteriovenous joint is disposed in a position where the artery channel abuts the vessel channel.

12. The vascular access device of claim 1, wherein an angle between a vertical axis perpendicular to the vascular access aperture and an axis running through center points of the vessel channel and the artery channel is greater than 45 degrees.

13. The vascular access device of claim 1, wherein an angle between a vertical axis perpendicular to the vascular access aperture and an axis running through center points of the vessel channel and the artery channel is between 30 degrees and 45 degrees.

14. The vascular access device of claim 1, wherein an angle between a vertical axis perpendicular to the vascular access aperture and an axis running through center points of the vessel channel and the artery channel is less than 30 degrees.

15. The vascular access device of claim 1, wherein the arteriovenous joint is offset from the vascular access aperture.

16. A method of using the vascular access device of claim 1 comprising:
creating an incision to insert the vascular access device;
positioning the partial artery channel of the bottom portion of the vascular access device around the bottom half of the artery and the partial vessel channel of the bottom portion of the vascular access device around the bottom half of the vessel;
positioning the partial artery channel of the top portion of the vascular access device around the top half of the artery and the partial vessel channel of the top portion of the vascular access device around the top half of the vessel to couple the top portion to the bottom portion;
closing the incision used to insert the vascular access device and allowing tissue surrounding the vascular access device to heal; and
after the incision is healed, creating an AV fistula between the artery and the vessel without uncoupling the top portion of the vascular access device from the bottom portion of the vascular access device.
